(19) United States
(12) Patent Application Publication
(10) Pub. No.: US 2016/0195510 A1
(43) Pub. Date: Jul. 7, 2016

(54) METHODS FOR DETERMINATION OF TOTAL HOMOCYSTEINE

(71) Applicant: Castle Medical, LLC, Smyrna, GA (US)

(72) Inventors: Rahul Nahire, Atlanta, GA (US); Thomas David, Atlanta, GA (US)

(21) Appl. No.: 14/985,778

(22) Filed: Dec. 31, 2015

Related U.S. Application Data
(60) Provisional application No. 62/098,846, filed on Dec. 31, 2014.

(51) Int. Cl. G01N 33/487; H01J 49/00

(52) U.S. Cl. CPC G01N 33/487; H01J 49/0031; H01J 49/004

(57) ABSTRACT

Disclosed herein are simple, rapid and cost effective methods for determination of endogenous sulfhydryl amino acid species from a sample, example a sample in clinical practice. The method can include mixing of a biological sample with an internal standards which does not require pretreatment or derivatization and/or isotopic dilution. The method can further include analyzing the sample using liquid chromatography tandem mass spectrometer (LC-MS/MS) to determine the concentration of sulfhydryl amino acid species.

### FIELD

The present disclosure relates to highly selective methods for detection and determination of one or more different endogenous sulfhydryl amino acid species for e.g. total homocysteine (tHcy) from a sample.

### BACKGROUND

Homocysteine (Hcy), an endogenous sulfhydryl amino acid generated by demethylation of methionine, is reported to have vascular endothelial cytotoxicity. It is one risk factor for the development of coronary heart disease independent from the other risk factors. Homocysteine levels have gained attention as many clinical studies have suggested that these levels are related to several clinical disorders (pregnancy complications, neural tube defects, mental disorders, psychogeriatric trouble, cancer, hyperinsulinemia) and most importantly cardiovascular diseases. Plasma homocysteine levels are associated with an increased risk of deep vein thrombosis (DVT) and pulmonary embolism (PE). Most of the studies have proved a strong, dose dependent and positive association between moderate hyperhomocysteinemia and risk of cardiovascular diseases especially atherosclerotic and atherothrombotic vascular disease. Determination of total homocysteine (tHcy) in plasma has become an important diagnostic procedure in clinical chemistry due to strong evidence supporting the hypotheses that increased homocysteine is an independent determinant of vascular disease and that hyperhomocysteinemia often may be amenable to homocysteine-lowering treatment with B vitamins. Recent studies have suggested that increased tHcy may play a role in the development of vascular complications of diabetes, and is a strong, independent risk factor for the development of dementia and Alzheimer's disease.

Once formed, Hcy is either irreversibly converted to cysteine or remethylated to methionine. Methionine may be regenerated by remethylation pathway, with help of methionine transferase (MS). All these reactions are depended of the amount of the methylenetetrahydrofolate, which is under control of another enzyme, the methylenetetrahydrofolate reductase (MTHFR) and the cofactor methylcobalamine (Vitamin B12).

The normal plasma homocysteine concentration ranges from 5 to 15 μM, with mean level about 10 μM Its higher level is described as hyperhomocysteinemia which can be moderate (15-30 μM), intermediate (30-100 μM) or severe (>100 μM). Elevated levels of Hcy can be a result of inherited disorders of either methionine transferase and/or methylenetetrahydrofolate reductase or it can be the result of deficiency of vitamin co-factors (B6, B12 and folate), homocysteine metabolic enzymes. Moderate hyperhomocysteinemia is caused by a decrease in the metabolic enzyme activity due to abnormality of genes, renal insufficiency, aging, smoking, lack of exercise or the like (Jacobsen, Clin. Chem. 44:8(B), 1998). Other reasons can be medications (phenytoin, carbamazepine, nitrous oxide, theophylline, metformin, colestipol, niacin, Penicillamine, thiazide, methotrexate, diuretics etc.), physiological factors (Age, Sex) and diseases (malignancies, anemia, hyperthyroidism, diabetes, chronic renal failure).

Most of the homocysteine in blood (99%) is present in the form of oxidized disulfide compounds (such as complex with protein, homocystine, cysteine-homocysteine) (Jacobsen, Clin. Chem. 44:8(B), 1998).

Several methods are available in the literature for Hcy determination in body fluids. Among these methods some are obsolete, like radio-enzymatic determination, whereas immunoassay and chromatographic methods are popular as being fully automated for routine analysis. Prior art analytical methods including chromatographic, capillary electrophoresis (CE), enzymatic-immunoassay and electrochemical methods are available for the determination of thiols in clinical and environmental samples. All methods, except those based on electrochemical and tandem-mass spectrometry detection, involves pre- or post-column derivatization of thiols.

Determination methods of homocysteine based on an immunoassay are disclosed in Japanese Patent Publication No. 9512634 and 10114797. Japanese Patent Publication No. 9512634 discloses immunological determination of Hcy by chemically modifying the Hcy to enhance its antigenicity. This technique is complicated and requires a large number of processe steps. Japanese Patent Publication No. 10-114797 discloses a method for direct determination of albumin bound Hcy only, and does not determine the total homocysteine (tHcy). Determination by immunoassay is relatively simple, but due to poor specificity it is difficult to meet the needs of high-level clinical research. Also such prior art methods have problems of cross-reactivity and interference from other endogenous substances which can lead to false results.

Determination methods of homocysteine based on a biochemical analysis are proposed in Japanese Patent No. 2870704. The method is based on treating homocysteine in a sample with a reducing agent and contacting with adenosine and S-adenosyl-L-homocysteine hydrolase and evaluating the amount of adenosine in the residual mixture. However, in this method, an inhibitor of the S-adenosyl-L-homocysteine hydrolase is not used, and therefore it is necessary to perform determination in kinetic mode. Furthermore, this method has the problem that because of presence of reducing agent, the produced hydrogen peroxide cannot be reacted with commonly used oxidative color-developing agent, which is an essential step for determining the total homocysteine. Therefore, an automatic analysis apparatus for general purposes cannot be used. However, the patent specification fails to disclose any method to avoid these problems.

Japanese Patent Publication No. 2000502262, U.S. Pat. Nos. 5,998,191 and 5,885,767 are based on reaction of Hcy with enzymes viz; homocysteine desulfurase, homocysteinase, or methionine-γ-lyase to detect the produced hydrogen sulfide, ammonia, or 2-oxobutyric acid. However, these methods have problems such as: (a) large number of process steps, (b) use of a lead ion, which is a harmful heavy metal, for the detection of the hydrogen sulfide; and (c) being affected by cysteine and methionine, which are structural analogs to homocysteine and present in a biological sample in a higher amount than that of homocysteine, making these methods less feasible for routine analysis.

A widely used technique for measuring total plasma Hcy is reversed-phase HPLC with fluorescence detection after derivatization of plasma thiols (Pastore A. et al., Clin Chem 1998; 44:825-32.). Some studies use gas chromatography-mass spectrometry techniques (Pietzsch J. et al., Clin Chem 1997; 43:2001-4.) with various derivatization protocols.

HPLC-based procedures and a fluorescence polarization immunoassay (FPIA) are the two methods most widely applied in clinical practice. Despite automation of sample preparation, HPLC method requires a visual verification of each profile by the operator and frequent manual corrections of baseline points at low concentrations. Inaccurate quantification because of interfering peaks or incomplete separation is a constant concern. The lack of internal standard and the relatively long instrument cycle time (17 min) are additional negatives. Although the FPIA is fully automated and faster than most HPLC methods, it could be affected by reagent batch variability and conceivably by other factors, and is not applicable to urine samples. Because urinary tHcy excretion has been reported to mirror plasma concentrations noninvasive determination of urinary tHcy could be regarded as the method of choice not only to monitor patients with homocystinuria but also as a tool to establish reliable pediatric reference ranges and to longitudinally study the impact of abnormal tHcy concentrations on cardiovascular disease from birth to adult life.

Thus, the conventional methods of determining homocysteine have problems such as requiring a special apparatus, complicated operation, insufficient sensitivity and specificity, therefore there is need of development of rapid and simple method of determining trace concentration of homocysteine with high sensitivity and specificity.

Nowadays LC-MS-MS methods are getting more popular as they are having advantage of being more rapid, reliable, sensitive and selective. HPLC methods are often used to separate a wide variety of polar and non-polar compounds; as the solvent (or mobile phase) and stationary phase that can be used in HPLC may be selected from a wide array of possibilities based upon the flexibility of the technique and the columns useful therein. In fact, with careful selection of the mobile phase and stationary phase, most sample mixtures can be separated into well-resolved peaks or fractions, which can subsequently be subjected to further analysis.

Characterization of these fractions by methods such as mass spectrometry (MS), e.g., LC/MS, provides qualitative information about the analytes present in the sample. In this way, it is often possible to identify the specific molecular species generating an MS signal by discerning its molecular weight. As such, MS is considered to be a very well utilized tool in the simultaneous qualitative characterization of one or more compounds in a single sample.

Chromatographic methods have been the methods of choice to determine plasma tHcy concentrations. Most laboratories have used methods based on the derivatization of homocysteine with thiol-specific reagents such as monobromobimane or ammonium 7-fluorobenzo-2-oxa-1,3-diazole-4-sulfonate (SBDF), or by o-phthaldialdehyde derivatization of the primary amine group. The fluorescent Hcy adduct is then separated from other thiol-containing compounds by HPLC and quantified by fluorescence detection. Other methods to measure tHcy by gas chromatography-mass spectrometry (GC-MS) have also been reported in literature.

It has also been studies that LC-MS-MS analysis is the most cost effective method among all other available methods. There are few reports of LC-MS-MS analysis of Hcy but many of those involve use of highly expensive deuteriated internal standard which increases the overall cost of the analysis. Most of these methods have run time of 5 or more minutes which again increases the time of analysis. So there is a need for rapid and cost effective method of tHcy determination in body fluids.

The present disclosure provides rapid and cost effective methods of tHcy determination in samples, including body fluids such as plasma and urine. The methods developed are highly sensitive for determination of total homocysteine in plasma by reduction with a thiol compound, precipitation of proteins, separation using reversed-phase chromatography and detection using a tandem mass spectrometer. The methods of present disclosure are easy and reliable for quantification of total homocysteine in plasma, which can meet the large-scale requirement of clinical analysis.

The increased demand in clinical practice for measuring plasma tHcy raises the issue of developing new methods better suited to accommodate high testing volumes and faster turnaround time in clinical samples.

### SUMMARY

This summary is intended to introduce, in simplified form, a selection of concepts that are further described in the detailed description. This summary is not intended to identify key or essential features of the claimed subject matter, nor is it intended to be used as an aid in determining the scope of the claimed subject matter.

The disclosed subject matter relates to methods for detection and/or determination of one or more different endogenous sulfhydryl amino acid species from a sample. In some embodiments, the sample can be a body fluid. For example, methods for determination of peptide biomarkers of cardiovascular disease in a subject are provided.

The method can include mixing the sample with internal standard and reducing the sulfhydryl amino acid species in the sample by a thiol compound. In some embodiments, reducing the sulfhydryl amino acid species can include allowing the sample and the thiol compound to react at room temperature, thereby producing reduced sulfhydryl amino acid species. The method can further include precipitating one or more proteins in the sample to form a mixture. In some embodiments, the proteins can be precipitated by addition of a solvent. The method can also include centrifuging the mixture to obtain a clear supernatant liquid and diluting the supernatant liquid with a mobile phase to form a solution, the solution can then be analyzed using for example, liquid chromatography tandem mass spectrometer (LC-MS-MS) to determine the sulfhydryl amino acid concentration present in the sample. In some embodiments, the method does not include derivatization and/or isotopic dilution of internal standard.

Described herein are methods for determination of endogenous sulfhydryl amino acid from the body fluid samples using LC-MS-MS. Also described are methods for determination of total homocysteine (tHcy) from a body fluid sample using LC-MS-MS. Further, methods for determination of total homocysteine (tHcy) without pretreatment or derivatization are described. Methods for determination of total homocysteine (tHcy) without the use of deuteriated internal standard are described. Methods for determination of homocysteine from a body fluid sample using LC-MS-MS are also described. Methods for determination of homocysteine without pretreatment or derivatization are described. Methods for determination of homocysteine without the use of deuteriated internal standard are described.

Methods for determination of total homocysteine (tHcy) and/or homocysteine that are less expensive and less time consuming are described. For example, the methods can have a run time of less than 5 minutes.

Additional advantages of the disclosed compositions and methods will be set forth in part in the description which follows, and in part will be obvious from the description. The advantages of the disclosed compositions will be realized and attained by means of the elements and combinations particularly pointed out in the appended claims. It is to be understood that both the foregoing general description and the following detailed description are exemplary and explanatory only and are not restrictive of the disclosed compositions, as claimed.

### BRIEF DESCRIPTION OF THE DRAWINGS

The accompanying Figures, which are incorporated in and constitute a part of this specification, illustrate several aspects described below.

FIG. 1 are graphs showing a calibration curve and a regression curve for tHcy.

FIG. 2 are graphs showing representative peaks (Qualifier and Quantifier) of Hcy and the internal standard pennicillamine.

FIG. 3 are chromatograms showing calibrator peaks with the internal standard.

FIG. 4 are chromatograms showing peaks of Liquicheck Hcy control (Levels 1, 2, and 3).

FIG. 5 are chromatograms showing the lower limit of detection and the lower limit of quantification of the calibration solutions.

### DETAILED DESCRIPTION

The materials and methods described herein can be understood more readily by reference to the following detailed description of specific aspects of the disclosed subject matter and the Examples and Figures included therein.

Before the present materials and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

### DEFINITIONS

The term “total homocyst(e)ine” refers to the sum of the concentrations of free homocysteine, protein-bound homocysteine, the disulfide homocystine, and the mixed disulfide homocysteine-cysteine. In some embodiments, homocysteine in a sample can be converted to reduced homocysteine by a reducing agent to determine the total homocysteine.

The term “accuracy” is art-recognized and describes the degree of conformity of a measure, i.e., the quantity, to a standard or a true value. For example, an increase in the accuracy of analyte quantification refers to an improvement in obtaining a measured value that is closer to the actual or true value. This improvement may be identified/described by reference to a percent increase in accuracy with respect to the accuracy obtainable using existing methods of measurement.

The term “amino acid” describes both naturally occurring and non-naturally occurring amino acids as well as amino acid analogs and mimetics.

The term “analyte” refers to any chemical or biological compound or substance that is subject to the analysis of the disclosure. Analytes can include, but are not limited to, small organic compounds, amino acids, peptides, polypeptides, proteins, nucleic acids, polynucleotides, biomarkers, synthetic or natural polymers, or any combination or mixture thereof.

The term “analyte derivative,” refers to an analyte that is functionalized with another moiety in order to convert the analyte into a derivative thereof. The analyte derivative can be detected for use in determining the unknown quantity of an analyte in a sample, using a response factor calculation.

The term “analyzing” or “analysis” refers to a method by which the quantity of each of the individual analytes described herein is detected. Such analysis may be made using any technique that distinguishes between the analyte (or analyte derivative) and the analyte standard (or analyte derivative standard). In some embodiments, the analysis or act of analyzing can include liquid chromatography-tandem mass spectrometry (LC-MS-MS).

The term “chromatographic separation” is art-recognized, and describes a process in which a chemical mixture carried by a liquid or gas is separated into components as a result of differential distribution of the solutes as they flow around or over a stationary liquid or solid phase. For example, chromatographic separations suitable for use in this disclosure can include, but are not limited to liquid chromatographic (including HPLC) methods such as normal-phase HPLC, RP-HPLC, HILIC, and size-exclusion chromatography (SEC), including gel permeation chromatography (GPC). Other suitable methods include additional HPLC methods and related liquid chromatographic techniques, including, e.g., ultra-performance liquid chromatography (HPLC), fast performance liquid chromatography (FPLC) and the like.

The term “internal standard,” refers to a collection of one or more functionalized chemical or biological compounds or substances, e.g., one or more analytes functionalized with another moiety in order to convert such compounds or substances into a derivative thereof. The internal standards are present in known concentrations and added to the sample to form a sample mixture. The addition of the internal standard allows for the detection of and comparison between the known concentrations of one or more known analytes, with the unknown concentrations of analytes in the original sample. As such, the internal standards can provide a way to measure the absolute quantity of an analyte in sample using a response factor calculation.

The term “liquid chromatography” is art-recognized and includes chromatographic methods in which compounds are partitioned between a liquid mobile phase and a solid stationary phase. Liquid chromatographic methods are used for analysis or purification of compounds. The liquid mobile phase can have a constant composition throughout the procedure (an isocratic method), or the composition of the mobile phase can be changed during elution (e.g., a gradual change in mobile phase composition such as a gradient elution method).

The term “mass spectrometry” and “mass spectroscopy” are art-recognized and used interchangeably to describe an instrumental method for identifying the chemical constitution of a substance by means of the separation of gaseous ions according to their differing mass and charge. A variety of mass spectrometry systems can be employed to analyze the analyte molecules of a sample subjected to the disclosed methods. For example, mass analyzers with high mass accuracy, high sensitivity and high resolution may be used and include, but are not limited to, atmospheric chemical ionization (APCI), chemical ionization (CI), electron impact (EI), fast atom bombardment (FAB), field desorption/field ionization (FD/FI), electrospray ionization (ESI), thermospray ionization (TSP), matrix-assisted laser desorption (MALDI), matrix-assisted laser desorption time-of-flight (MALDI-TOF) mass spectrometers, ESI-TOF mass spectrometers, and Fourier transform ion cyclotron mass analyzers (FT-ICR-MS). In addition, it should be understood that any combination of MS methods could be used in the methods described herein to analyze an analyte in a sample. In certain embodiments, the MS technique used for analysis of the analyte described herein is one that is applicable to most polar compounds, including amino acids, e.g., ESI.

The term “mobile phase” is art-recognized, and describes a solvent system (such as a liquid) used to carry a compound of interest into contact with a solid phase (e.g., a solid phase in a solid phase extraction (SPE) cartridge or HPLC column) and to elute a compound of interest from the solid phase.

The term “precision” is art-recognized and describes the reproducibility of a result. It is measured by comparison of successive values obtained for a measurement to the prior values, where more precise measurements (or those with greater precision) will be demonstrated by successive measurements that are more consistently closer to the prior measurements.

The terms “quantitative” and “quantitatively” are art-recognized and refers to measurements of quantity or amount. For example, the term “quantification” describes the act of measuring the quantity or amount of a particular object, e.g., an analyte. In some embodiments, the quantitative analysis can be a measurement of an absolute amount, as opposed to relative amount, i.e., the total amount of analyte may be quantified absolutely in order to determine the actual amount of the analyte.

The term “sample” refers to a representative portion of a larger whole or group of components that are capable of being separated and detected by the disclosed methods. Exemplary samples include chemically or biologically derived substances, e.g., analytes of the disclosed methods. In particular embodiments, the components of the sample include, but are not limited to small organic compounds, amino acids, peptides, polypeptides, proteins, nucleic acids, polynucleotides, biomarkers, synthetic or natural polymers, or any combination or mixture thereof.

The term “sample mixture,” refers to the resultant product when a sample is mixed or combined with one or more analyte derivative standards, e.g., of a known concentration.

The disclosed subject matter relates to methods for detection and/or determination of one or more different endogenous sulfhydryl amino acid species from a sample. In some embodiments, the endogenous sulfhydryl amino acid species can include homocysteine, cysteine, cystine, methionine, and combinations thereof. In some examples, the sulfhydryl amino acid species is homocysteine. The sample can be a body fluid. For example, the sample can be a bodily fluid selected from the group consisting of oral fluids (saliva), sweat, urine, blood, serum, plasma, spinal fluid, and combination thereof.

The method can include mixing the sample with an internal standard, and reducing the sulfhydryl amino acid species in the sample by a thiol compound. Reducing the sulfhydryl amino acid species can include allowing the sample and the thiol compound to react at room temperature thereby producing reduced sulfhydryl amino acid species. The method can also include precipitating one or more proteins from the reduced sulfhydryl amino acid sample to form a mixture. In some embodiments, precipitating the one or more proteins can include addition of a solvent. The method can also include centrifuging the mixture to obtain a supernatant liquid. The supernatant can be transferred to a vial, such as an auto-sampler vial for analysis. The method can include diluting the supernatant liquid with a mobile phase to form a solution and analyzing the solution. Analysis can be done using liquid chromatography tandem mass spectrometer (LC-MS-MS) to determine the sulfhydryl amino acid concentration present in the sample. In some embodiments, the method is devoid of derivatization and/or isotopic dilution of internal standard.

In some examples, highly selective and specific methods for detection or determination of the total homocysteine (tHcy) in a sample can include the steps: (a) mixing the sample with an internal standard, (b) reducing the homocysteine in the sample by a thiol compound, (c) precipitating proteins in the sample for example, by the addition of solvent, (d) centrifuging of the mixture of step (c) to obtain a clear supernatant liquid, (e) diluting the clear supernatant liquid with mobile phase to form a solution, and (f) analyzing the solution using for example, liquid chromatography tandem mass spectrometer (LC-MS-MS) to determine the total homocysteine (tHcy) present in said sample. In some embodiments, the method is devoid of derivatization and/or isotopic dilution of internal standard.

In some examples, highly selective and specific method for detection or determination of homocysteine (Hcy) in a sample can include the steps: (a) mixing the sample with an internal standard, (b) reducing the homocysteine in the sample by a thiol compound, (c) precipitating proteins in the sample for example, by the addition of solvent, (d) centrifuging of the mixture of step (c) to obtain a clear supernatant liquid, (e) diluting the clear supernatant liquid with mobile phase to form a solution, and (f) analyzing the solution using for example, liquid chromatography tandem mass spectrometer (LC-MS-MS) to determine the total homocysteine (tHcy) present in said sample. In some embodiments, the method is devoid of derivatization and/or isotopic dilution of internal standard.

The internal standard can be selected from the group of compounds whose structure is closely related to homocysteine such as cysteamine, cysteine, cystine, methionine and penicillamine. Table 1 shows the structure of exemplary internal standards.

TABLE 1
Structures of exemplary internal standards.

| | |
|---|---|
| Homocysteine | 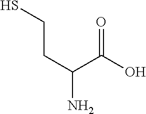 |
| Penicillamine | 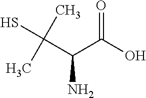 |
| Cysteamine | 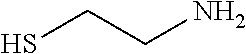 |
| Cysteine | 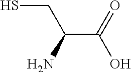 |
| Cystine | 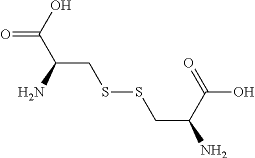 [SMILES: N[C@H](CSSC[C@H](N)C(=O)O)C(=O)O] |
| Methionine | 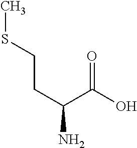 |
| Total homocysteine (tHcy) | 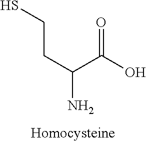 |
| | 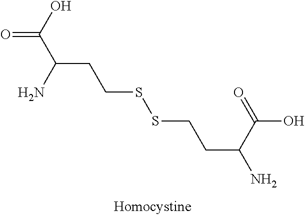 [SMILES: NC(CCSSCCC(N)C(=O)O)C(=O)O] |
| | 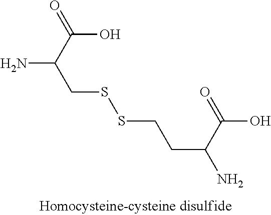 [SMILES: NC(CCSSCC(N)C(=O)O)C(=O)O] |

The proteins can be precipitated using any suitable method. In some embodiments, precipitating the proteins include the use of a salting-out salts, polymers, and organic solvents (a solvent and/or co-solvents) for precipitation of proteins. Suitable salts can include ammonium sulfate. Suitable polymers can include high molecular weight polyethylene glycol. Suitable organic solvents can include various alcohols such as methanol and ethanol, acetone and dioxane.

In some embodiments, the liquid chromatography tandem mass spectrometer (LC-MS-MS) can include matrix-assisted laser desorption/ionization (MALDI) time-of-flight (TOF) MS analysis or electrospray ionization (ESI) MS. For example, the amount of homocysteine in a sample can be determined using liquid chromatography electrospray tandem mass spectrometry (LC-MS-MS). In some embodiments, the transitions for homocysteine in mass spectra with tandem mass spectrometer are at 136 to 90 and 136 to 118.

In some embodiments, the tHcy can be isolated from a fraction of the sample, for example a bound fraction or an unbound fraction. In some embodiments, the bound fraction is an albumin-bound fraction or an immunoglobulin-bound fraction.

The methods can include the use of suitable releasing agents capable of liberating the tHcy and Cys sulfur compounds from serum proteins. Reducing agents can include sodium and potassium borohydride; thiols such as dithiothreitol, dithioerythritol, 2-mercaptoethanol, thioglycolic acid and glutathione; and phosphines and trialkylphosphines such as tributylphosphine and tris(2-carboxyethyl)phosphine. Particularly suitable reducing agents are dithiothreitol and tris(2-carboxyethyl)phosphine (TCEP).

In some embodiments, the sample, such as a saliva sample, a blood sample, a serum sample, a plasma sample, or a urine sample can be obtained from a subject. In some aspects, the subject is a human. In some embodiments, the subject is a diabetic subject. In some embodiments, the subject has a cardiovascular disease such as a coronary artery disease (CAD) (e.g., atherosclerosis), a peripheral vascular disease, or both.

Methods for detecting and/or determining the amount of homocysteine in a urine sample are provided. For example, the method can include treating the urine sample with a thiol compound to form a mixture and allowing the mixture to react at room temperature. The method can also include preparing calibrators using synthetic urine. The homocysteine can be detected or the amount determined using LC-MS-MS.

Methods for detecting and/or determining the amount of total homocysteine in plasma and/or urine samples by electrospray tandem mass spectrometry are provided. The method can include determining the amount of at least one peptide in the sample using mass spectrometry (MS) analysis or electrospray ionization (ESI) MS).

The present methods can have one or more advantages. For example, the methods can have one or more of the following advantages:

1. The methods can have two transitions, 136 to 90 and 136 to 118, compared to other methods in literature which have only one transition 136-90.

2. The methods do not require pretreatment or derivatization.

3. The methods do not use expensive deteuriated internal standard.

4. The methods are highly selective towards determination of tHcy.

5. The methods have high throughput potential.

6. The methods are less expensive and less time consuming.

7. The methods have less margin for introducing variations and this prevents errors into the procedure of determination of homocysteine.

8. The methods include a simple and rapid 2 minutes of run time.

### Examples

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures and other reaction ranges and conditions that can be used to optimize the product purity and yield obtained from the described process. Only reasonable and routine experimentation will be required to optimize such process conditions.

Reagents: DL-homocysteine, L-penicillamine, DL-dithiothreitol were purchased from Sigma Aldrich. Formic acid and ammonium formate were purchased from Sigma Aldrich and were of analytical standard. Deionized water, acetonitrile and methanol were HPLC grade. Human serum was purchased from Utak laboratories. All other chemicals and solvents were of the highest purity available from commercial sources and used without further purification.

Determination of tHey: A serum or plasma sample (100 mL) was mixed with 50 mL of internal standard solution (75 μmol). Complete reduction of disulfides was accomplished by the addition of 20 μL of 50 mM dithiothreitol, which was allowed to react at room temperature for 15 min. Proteins were precipitated by the addition of 100 μL of methanol. After 20 min centrifugation at 15000 rpm, 100 μL of the clear supernatant was transferred to an auto-sampler vial, diluted with mobile phase-A (1:4). Calibrators were prepared in plasma of known tHcy concentration by the addition of a 200 μmol/L working solution corresponding to tHcy concentrations of 5, 10, 25, 50, and 100 μmol/L.

For urine samples, simple dilute and shoot method was employed. Samples were treated with 20 μL of 50 mM dithiothreitol, which was allowed to react at room temperature for 15 min. Calibrators were prepared in synthetic urine by the addition of a 200 μmol/L working solution corresponding to tHcy concentrations of 1, 5, 10, 25, 50, and 100 μmol/L.

Two in-house controls (10-200) and three out-house controls (5-15-40) were used.

LC-MS set up: ABSciex-Triple Quadrupole System 4500 consists of an ion source, enhanced desolvation technology with Electron Spray Ionization in positive mode.

For all MS-MS experiments, mass calibration and resolution adjustments (at 0.7 amu full width at half height) on both the resolving quadrupoles were automatically optimized using a poly(propylene)glycol 1 3 1024 mol/L solution introduced via the built-in infusion pump. In the ABSciex API3200 Triple Quad adjustable voltage, Declustering Potential (DP) declusters ions, Entrance Potential (EP) focuses ions, Collision Cell Entrance Potential (CEP) focuses ions into Q2, Collision Energy fragments ions and Collision Cell exit potential (CXP) assists ions going into Q3. All these voltages are optimized for each analyte in the assay by compound optimization and the values are incorporated into the acquisition method.

Tables 2 and 3 show the chromatography and ion source parameters, respectively.

TABLE 2
Chromatography parameters
Chromatography parameters

| | |
|---|---|
| Chromatography mode | Reverse phase |
| Isocratic/gradient method | Isocratic |
| Aqueous phase | DI H₂O with 0.1% Formic Acid and 0.1% |
| | Ammonium Formate |
| Organic phase | 50.0% Acetonitrile and 50.0% Methanol with |
| | 0.1% Formic Acid |
| Needle Wash | 40.0% Isopropanol + 40.0% Acetonitrile + |
| | 20.0% Acetone |
| Flow rate | 1 mL/min |
| Run time | 2 minutes |
| Sample injection volume | 10 μL |
| Column temperature | 45° C. |

TABLE 3
Ion source parameters
Ion source parameters

| Interface | Electrospray ionization |
|---|---|
| Ionization mode | Positive |
| Source/Gas temperature | 550° C. |
| Ion source gas 1 | 60 PSI |
| Ion source gas 2 | 60 PSI |
| Curtain Gas | 35 PSI |
| Collision gas | 7 PSI |
| Ion Spray voltages | 2500 |

List of parent ions and fragment ions with their optimized declustering potentials (DP), entrance potentials (EP), collision energies (CE) and collision cell exit potentials (CXP) for each transition in Positive Mode.

The present methods are based on the principle of operation of an ESI-MS-MS system where, ESI generates charged ions at near-atmospheric pressure from a solution nebulized as a fine spray of droplets by a high voltage electric field and/or pneumatically. Ion desorption from the droplets is induced by a counter flow of gas, and a fraction of the charged molecules is drawn in to the high vacuum of the quadrupole through a narrow opening. The first (Q1) or third (Q3) quadrupoles can be set either to scan a mass range or to select one or more individual ions. The second quadrupole (Q2) is used as a collision cell. When nitrogen is introduced into the Q2 region, fragmentation of ions passed or scanned through Q1 is enhanced by collisional activation, and the resulting fragment ions are then resolved by Q3. In the multiple reaction monitoring (MRM) mode, Q1 is set to transmit only the parent ions of interest (m/z 136 and m/z 150.3 for Hcy and penicillamine, respectively), Q3 scans only in the mass range of the daughter ions (m/z 90 and m/z 118) arising from the primary, collisionally activated fragmentation in Q2.

The results indicate that ESI-MS-MS provides an approach to the determination of tHcy in plasma and urine. Sample preparation is based on a simplified manual procedure (40 samples were prepared in 1 h including incubation time) that can be automated, uses inexpensive reagents and no derivatization and takes advantage of a stable isotope-labeled internal standard with identical chromatographic behavior, which also serves as internal control of the reduction step. No interference from other compounds present in either serum/plasma or urine samples were noted in the analysis of over 500 samples.

Method validation: The method was validated for linearity, accuracy, precision, recovery, LOD and LOQ.

Calibration curves for homocysteine, were made by serial dilution from a stock solution and were created in duplicates. Peak heights vs nominal concentrations were used to construe calibration curves. Curves were evaluated using least squares fitting and by linear regression analysis.

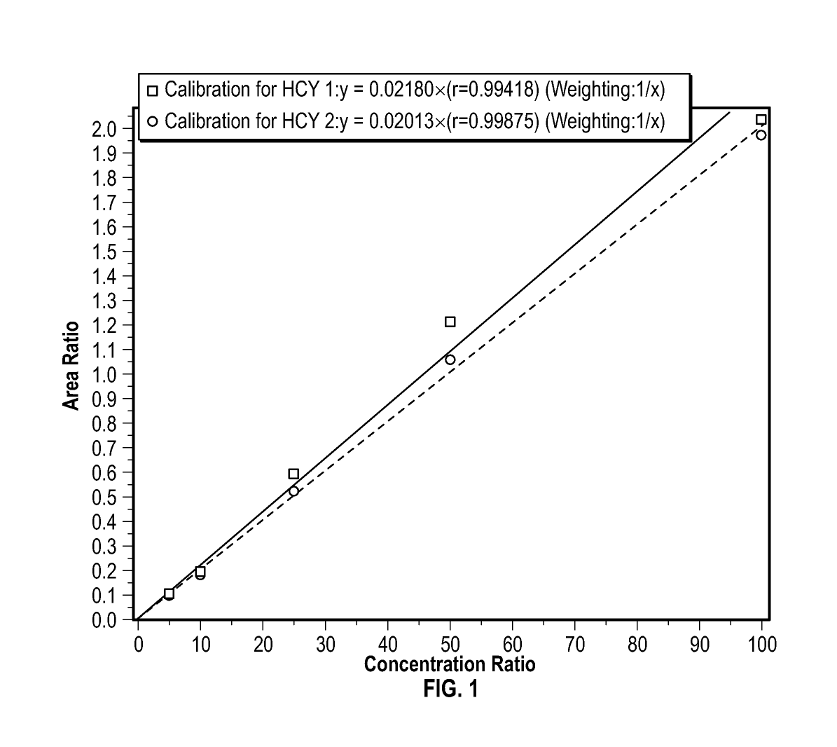

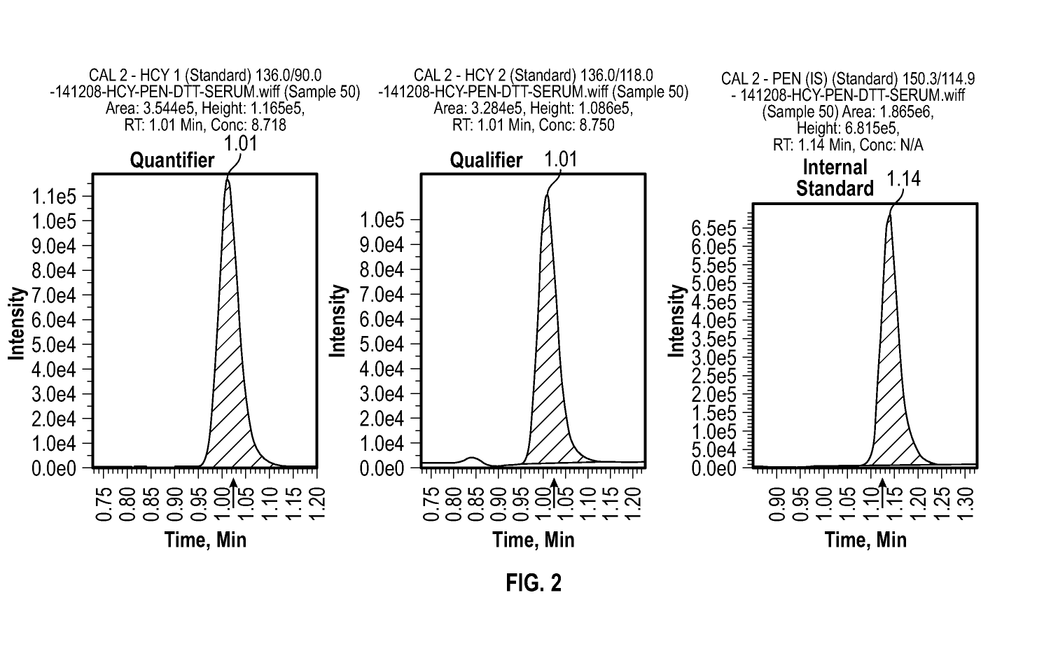

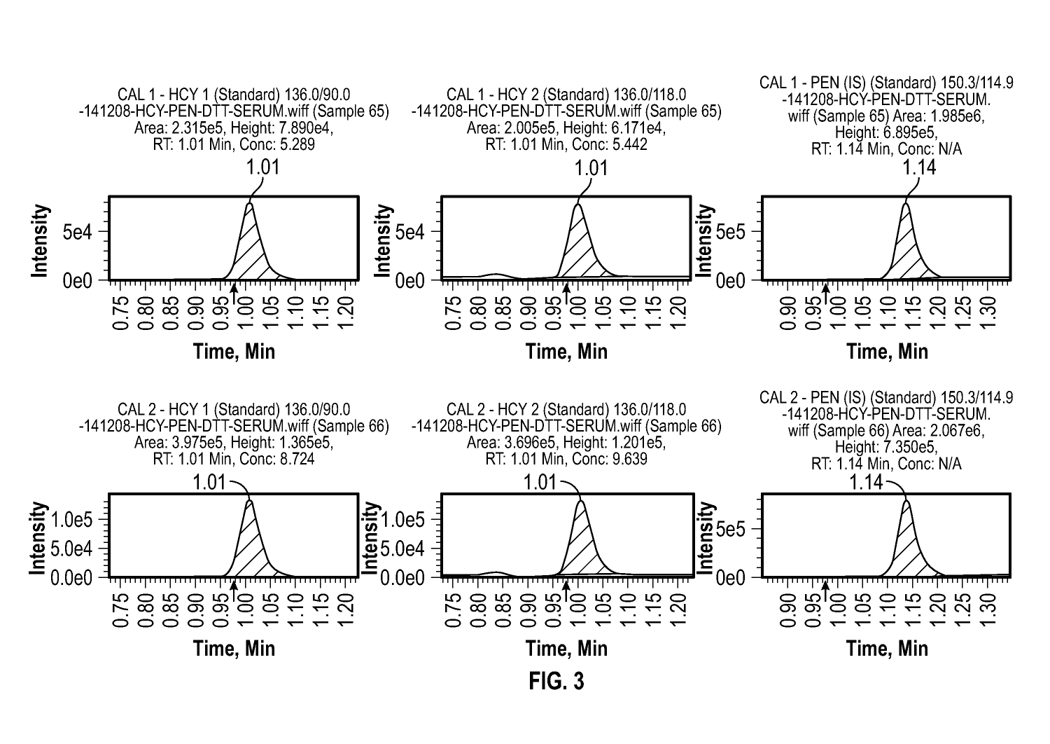

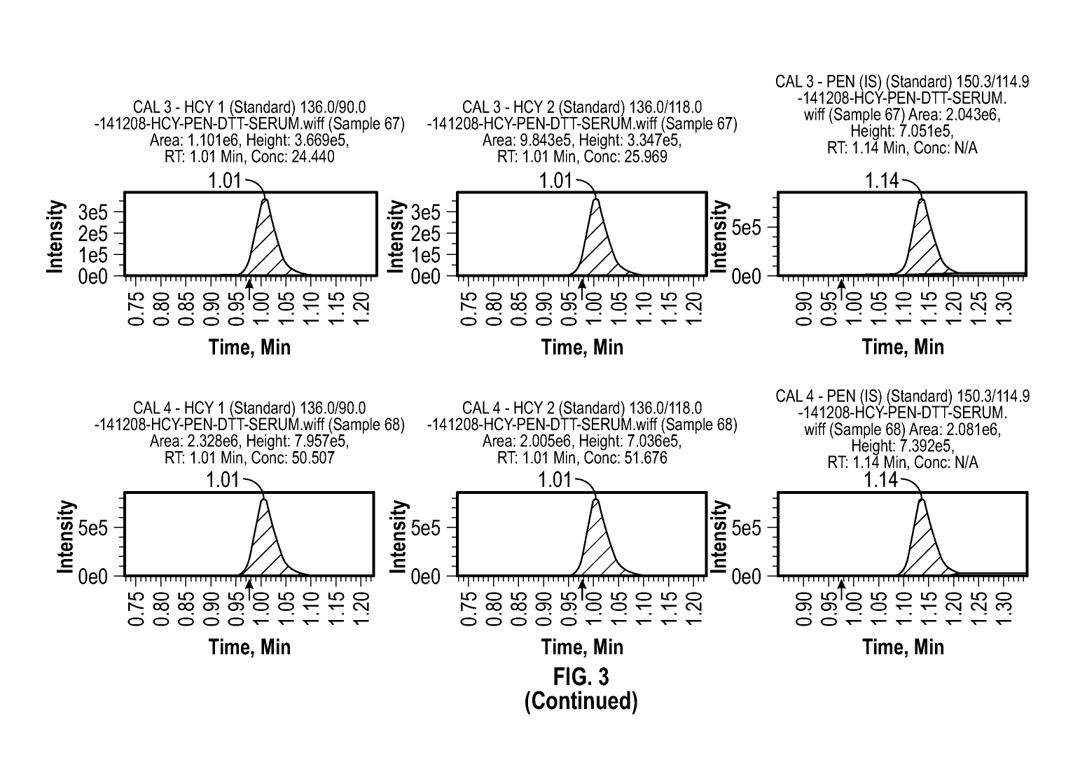

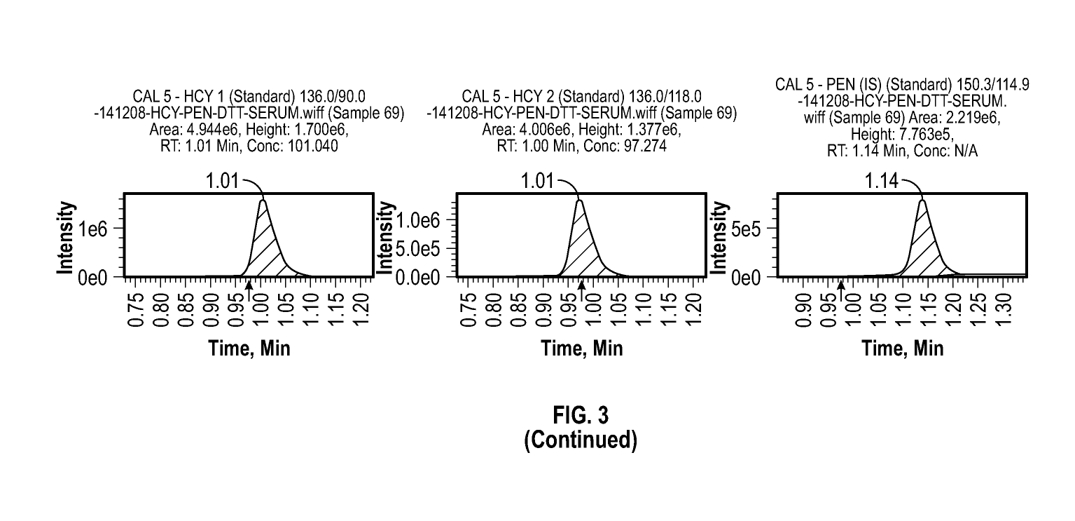

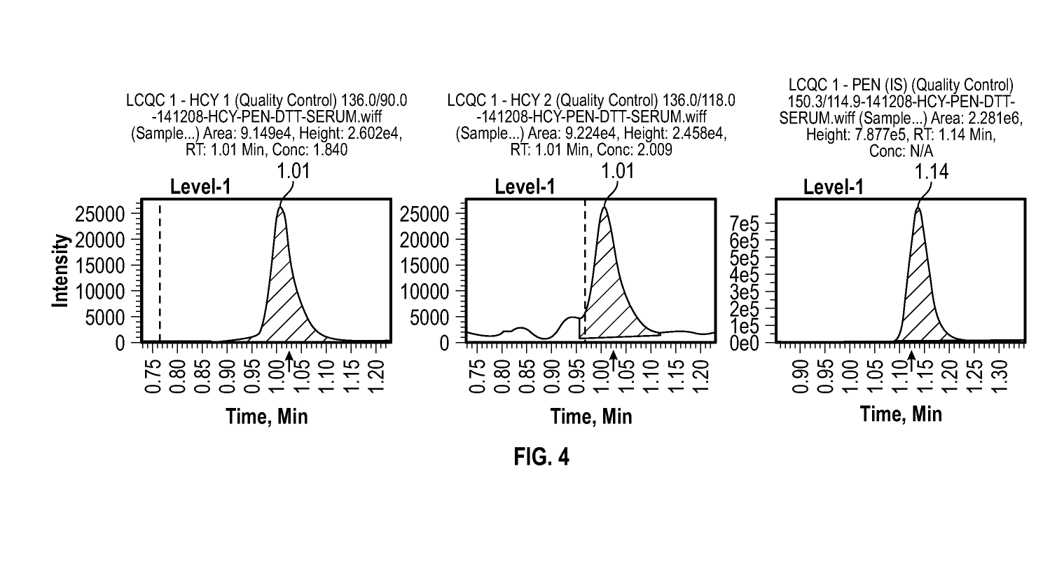

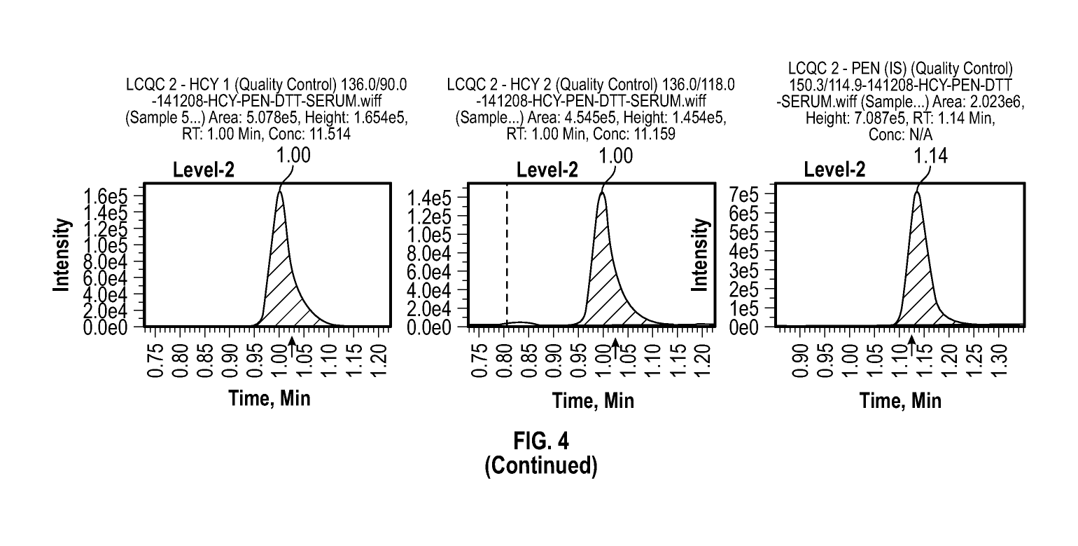

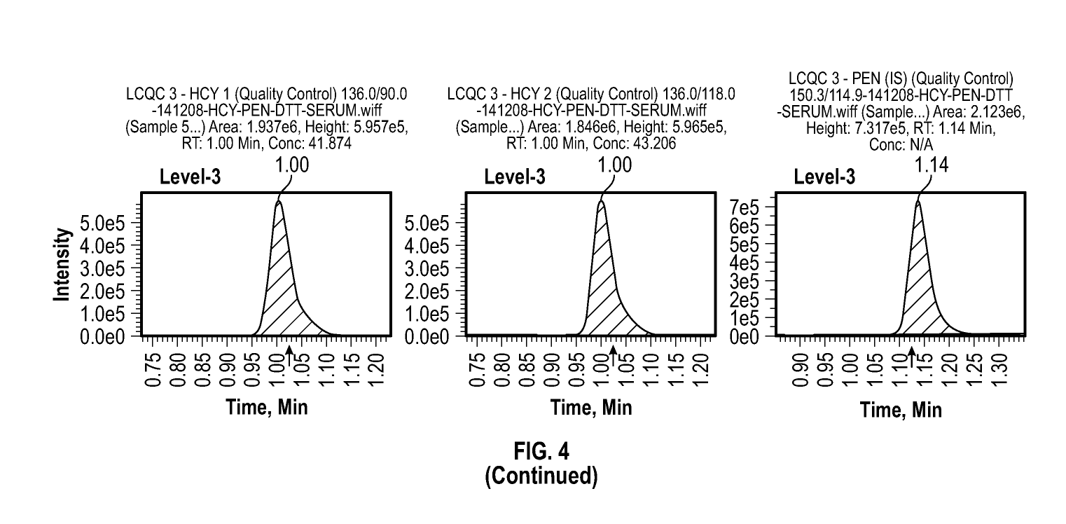

The calibration data obtained for concentrations from 2.5 μM/L to 100 μM/L is shown in FIG. 1.

Linearity: LOQ was established at 2.5 μg/mL and linearity was established from 5-10-25-50-100 μg/mL. Regression coefficient was 0.994 for Quantifier and 0.998 for Qualifier transitions.

Accuracy: Accuracy was done with five injections of three concentrations (5-10-25 μM/L). Standard deviation was found to be less than 5 and percent CV was less than 15. Table 4 shows an accuracy study of homocysteine.

TABLE 4
Accuracy studies for homocysteine
Accuracy studies for homocysteine

| Accuracy | Level 5 | Level 10 | Level 25 |
|---|---|---|---|
| Injection 1 | 5.9 | 8.6 | 30 |
| Injection 2 | 4.9 | 8 | 20 |
| Injection 3 | 4.2 | 7.3 | 23 |
| Injection 4 | 4.7 | 9.5 | 23 |
| Injection 5 | 4 | 8.7 | 23 |
| Avg | 4.74 | 8.42 | 23.8 |
| SD | 0.74364 | 0.8228 | 3.701351 |
| % CV | 15.6886 | 9.771973 | 15.5519 |

Precision: Intra-day and inter-day precisions were calculated by injecting calibrators and quality control samples on same day and different days. Percent CV was found be less than 15 for all the injections. The intra-day and intra-day precision data is summarized in Tables 5 and Table 6 respectively.

TABLE 5(a)
Intraday precision with calibrators
Precision: Calibrators

| Calibrators | Cal 1 | Cal 2 | Cal 3 | Cal 4 | Cal 5 |
|---|---|---|---|---|---|
| Injection 1 | 4.7 | 10.3 | 28.4 | 38.3 | 104 |
| Injection 2 | 4.7 | 8.7 | 27.3 | 55.7 | 93.5 |
| Injection 3 | 5.2 | 8.7 | 24.5 | 50.5 | 101 |
| Avg | 4.866667 | 9.233333 | 26.73333 | 48.16667 | 99.5 |
| SD | 0.288675 | 0.92376 | 2.010804 | 8.931592 | 5.408327 |
| % CV | 5.931681 | 10.00463 | 7.521711 | 18.5431 | 5.435504 |

TABLE 5(b)
Intraday precision with out-house quality controls
Precision: Quality Control (out house)

| Out House QC | QC Level 1 | QC Level 2 | QC Level 3 |
|---|---|---|---|
| Injection 1 | 2 | 14.6 | 44.4 |
| Injection 2 | 1.8 | 11.5 | 41.8 |
| Injection 3 | 1.8 | 13 | 48 |
| Avg | 1.866667 | 13.03333 | 44.73333 |
| SD | 0.11547 | 1.550269 | 3.113412 |
| % CV | 6.185896 | 11.89465 | 6.959937 |

TABLE 5(c)
Intraday precision with in-house quality controls
Precision: Quality Control (In House)

| In House QC | In House 1 | In House 2 |
|---|---|---|
| Injection 1 | 9.5 | 171 |
| Injection 2 | 6.9 | 199 |
| Injection 3 | 8.8 | 200 |
| Avg | 8.4 | 190 |
| SD | 1.3453624 | 16.4620776 |
| % CV | 16.0162191 | 8.66425139 |

TABLE 6(a)
Interday precision with calibrators
Precision: Calibrators

| | LOQ | Cal 1 | Cal 2 | Cal 3 | Cal 4 | Cal 5 |
|---|---|---|---|---|---|---|
| Injection 1 | 3.7 | 4.59 | 8.6 | 17.4 | 43.9 | 114 |
| Injection 2 | 2.1 | 4 | 6.6 | 23.4 | 43.2 | 113 |
| Injection 3 | 2.3 | 3.8 | 7.2 | 19.4 | 52.8 | 106 |
| Injection 4 | 2.9 | 4.5 | 7.5 | 22.7 | 46 | 108 |
| Injection 5 | 2.2 | 5.5 | 7.7 | 21 | 46 | 109 |
| Avg | 2.64 | 4.478 | 7.52 | 20.78 | 46.38 | 110 |
| SD | 0.669328 | 0.660621 | 0.732803 | 2.445813 | 3.800263 | 3.391165 |
| % CV | 25.35333 | 14.75259 | 9.744719 | 11.77003 | 8.193754 | 3.082877 |

TABLE 6(b)
Interday precision with out-house quality controls
Precision: Quality Controls (Out of House)

| | QC Level 1 | QC Level 2 | QC Level 3 |
|---|---|---|---|
| Injection 1 | 4.1 | 15.7 | 55 |
| Injection 2 | 3.2 | 16.3 | 51.6 |
| Injection 3 | 3.1 | 14.7 | 46 |
| Injection 4 | 4.2 | 17.3 | 50.9 |
| Injection 5 | 5.9 | 17.6 | 54 |
| Avg | 4.1 | 16.32 | 51.5 |
| SD | 1.124722 | 1.184061 | 3.504283 |
| % CV | 27.43225 | 7.255275 | 6.804433 |

TABLE 6(c)
Interday precision with in-house quality controls
Precision: Quality Controls (In House)

| | In House 1 | In House 2 |
|---|---|---|
| Injection 1 | 9.5 | 273 |
| Injection 2 | 8 | 233 |
| Injection 3 | 6.6 | 231 |
| Injection 4 | 8.2 | 248 |
| Injection 5 | 9.7 | 211 |
| Avg | 8.4 | 239.2 |
| SD | 1.258968 | 23.02607 |
| % CV | 14.98771 | 9.626284 |

Recovery: Recoveries were calculated by adding known concentrations of homocysteine, to 3 different samples previously analyzed, and then the final concentrations were measured in duplicate. Results were measured as differences between the measured and the theoretical values and expressed as percentage of recovery.

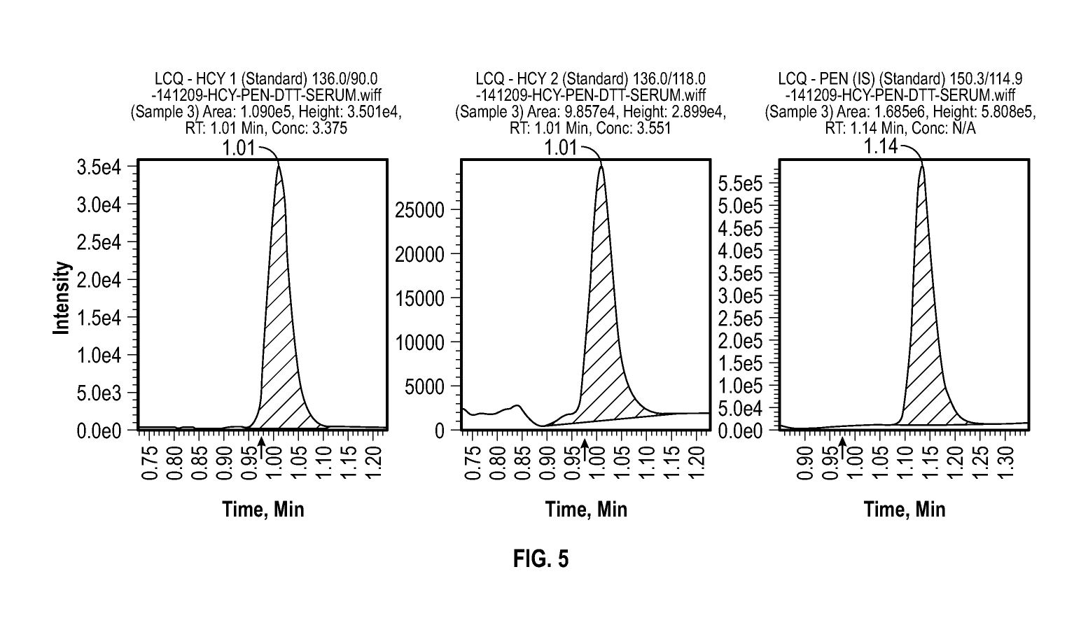

LOD & LOQ: The lower limit of detection (LOD) and the lower limit of quantification (LOQ), defined as the lower concentration giving a signal to noise ratio higher than 3 and 10, respectively, were obtained measuring in triplicate the calibration solutions. LOD was found to be 1 μM/L and LOQ was 2.5 μM/L (FIG. 5).

What is claimed is:

1. A method for detection or determination of one or more different endogenous sulfhydryl amino acid species from a sample, comprising:
a) mixing the sample with an internal standard;
b) reducing the sulfhydryl amino acid species in the sample by a thiol compound;
c) precipitating proteins in the sample to form a mixture;
d) obtaining a supernatant liquid from the mixture of step (c); and
e) analyzing said supernatant using liquid chromatography tandem mass spectrometer (LC-MS-MS) to determine the sulfhydryl amino acid concentration present in the sample;
wherein, the method does not include derivatization and/or isotopic dilution of the internal standard.

2. The method of claim 1, wherein the internal standard is selected from the group consisting of cysteamine, cysteine, cystine, methionine, and penicillamine.

3. The method of claim 2, wherein the internal standard is penicillamine.

4. The method of claim 1, wherein transitions for homocysteine in mass spectra with tandem mass spectrometer are at 136 to 90 and 136 to 118.

5. The method of claim 1, wherein the sample is a bodily fluid selected from the group consisting of saliva, sweat, urine, blood, serum, plasma, spinal fluid, and combinations thereof.

6. The method of claim 1, wherein the liquid chromatography tandem mass spectrometer (LC-MS-MS) comprises matrix-assisted laser desorption/ionization (MALDI) time-of-flight (TOF) MS analysis or electrospray ionization (ESI) MS.

7. A method for detection or determination of total homocysteine (tHcy) from a sample, comprising:
a) mixing the sample with an internal standard,
b) reducing the homocysteine in the sample by a thiol compound,
c) precipitating proteins in the sample to form a mixture,
d) obtaining a supernatant liquid from the mixture of step (c), and
e) analyzing said supernatant using liquid chromatography tandem mass spectrometer (LC-MS-MS) to determine the total homocysteine (tHcy) present in the sample;
wherein, the method does not include derivatization and/or isotopic dilution of the internal standard.

8. The method of claim 7, wherein the internal standard is selected from the group consisting of cysteamine, cysteine, cystine, methionine, and penicillamine.

9. The method of claim 8, wherein the internal standard is penicillamine.

10. The method of claim 7, wherein transitions for the homocysteine in mass spectra with tandem mass spectrometer are at 136 to 90 and 136 to 118.

11. The method of claim 7, wherein the sample is a bodily fluid selected from the group consisting of saliva, sweat, urine, blood, serum, plasma, spinal fluid, and combination thereof.

12. The method of claim 7, wherein the liquid chromatography tandem mass spectrometer (LC-MS-MS) comprises matrix-assisted laser desorption/ionization (MALDI) time-of-flight (TOF) MS analysis or electrospray ionization (ESI) MS.

13. A method for detection or determination of homocysteine (Hcy) from a sample of body fluid, comprising:
a) mixing the sample with an internal standard;
b) reducing the homocysteine in the sample by a thiol compound;
c) precipitating proteins in the sample to form a mixture;
d) obtaining a supernatant liquid from the mixture of step (c); and
e) analyzing said supernatant using liquid chromatography tandem mass spectrometer (LC-MS-MS) to determine the homocysteine (Hcy) present in said sample;
wherein, the method is devoid of derivatization and/or isotopic dilution of internal standard.

14. The method of claim 13, wherein the internal standard is selected from the group consisting of cysteamine, cysteine, cystine, methionine, and penicillamine.

15. The method of claim 14, wherein the internal standard is penicillamine.

16. The method of claim 13, wherein transitions for the homocysteine in mass spectra with tandem mass spectrometer are at 136 to 90 and 136 to 118.

17. The method of claim 13, wherein the bodily fluid is selected from the group consisting of saliva, sweat, urine, blood, serum, plasma, spinal fluid, and combinations thereof.

18. The method of claim 13, wherein the liquid chromatography tandem mass spectrometer (LC-MS-MS) comprises matrix-assisted laser desorption/ionization (MALDI) time-of-flight (TOF) MS analysis or electrospray ionization (ESI) MS.
